(19) United States
(12) Patent Application Publication
(10) Pub. No.: US 2003/0022250 A1
(43) Pub. Date: Jan. 30, 2003

(54) Functional IgE test methods and compositions

(76) Inventors: Stephen C. Dreskin, Denver, CO (US); Donald A. Dibbern JR., Tigard, OR (US); Phillip B. Williams, Olathe, KS (US)

Correspondence Address: GREENLEE, WINNER AND SULLIVAN, P.C., Suite 201, 5370 Manhattan Circle, Boulder, CO, 80303, US

(21) Appl. No.: 10/158,568

(22) Filed: May 29, 2002

Related U.S. Application Data
(60) Provisional application No. 60/294,465, filed on May 29, 2001.
(60) Provisional application No. 60/294,466, filed on May 29, 2001.

(51) Int. Cl. G01N 33/567

(52) U.S. Cl. 435/7.21

(57) ABSTRACT

The present assays provide ex vivo methods for determining a significant and potentially life threatening response of mast cells and/or basophils of a patient to one or more antigens, where the response is mediated by IgE and the FcεRI protein binding and serotonin, histamine or other signaling or mediator of IgE-allergen binding. Cells which express IgE and FcεRI are labeled by uptake of detectable 5-hydroxytryptamine (serotonin) or other signaling compound, and the release of the intracellular pool of detectable compound is triggered by the addition of the allergen to which sensitivity is observed. Significant release in response to a particular compound or composition indicates that there is potentially danger to the patient from whom the cells were prepared if that patient comes in contact with the allergen. The assay can also be manipulated to detect the presence of a potentially dangerous allergen in a composition. Significant release of the detectable signaling compound, especially radiolabeled 5-HT, in response to a composition or compound indicates that there is the potential for a dangerous response to the composition or particular allergen.

### CROSS REFERENCE TO RELATED APPLICATIONS

This application claims benefit of U.S. Provisional Application No. 60/294,465, filed May 29, 2001 and U.S. Provisional Application No.60/294,466, filed May 29,2001, both of which are incorporated by reference herein.

### ACKNOWLEDGMENT OF FEDERAL RESEARCH SUPPORT

not applicable

### BACKGROUND OF THE INVENTION

The present invention relates to the area of immunology, especially as applied to the diagnosis of allergies which can be life threatening, in particular those allergies which are associated with IgE recognizing a specific allergen, and the present invention further relates to immunological methods for the detection of particular allergens or antigens and/or the standardization of allergen extracts for particular allergens.

Current ex vivo or in vitro tests depend upon identification of allergens in binding assays (e.g. ELISA or RIA) that do not reflect the functionality of the interaction of the allergen-specific IgE with the allergen. There is a need in the art for a method that is both ex vivo or in vitro, does not depend on the presence of the patient or of functional cells from the patient, and measures a functional interaction between the allergen-specific IgE and the allergen in the same fashion that the in vivo skin test measures this functional interaction.

Certain individuals are genetically prone to inappropriate IgE-mediated diseases. There is no known common feature among the allergens and/or antigens to which certain patients respond The frequency, route and dosage of exposure all affect the strength of the response of a sensitive individual to a particular antigen or allergen. Known allergenic materials include, but are not limited to, peanuts and other common foods, pollen of various types including but not limited to ragweed, animal dander (which includes salivary proteins), insect and other venoms and insect parts and excrement, occupational materials such as latex, and drugs such as penicillin. Many of these sensitivities (allergies) are often associated with allergic responses which are systemic and can be life-threatening or even fatal.

There is a long felt need in the art for a test which is safe for the patient being tested and which predicts functionally important and severe IgE-mediated immune responses with confidence. There is a also a need for sensitive assays for the detection of allergens and antigens to which certain individuals exhibit an immunological response, especially IgE responses which are, in some individuals, life threatening. There is further need for methods to allow the standardization of the functional activities of allergen extracts for consistency in assay results from batch to batch. The present invention fulfills these needs.

### SUMMARY OF THE INVENTION

The present invention provides a method for assessing the functional interactions of allergens with human IgE, obtained from allergic individuals or engineered molecules that can combine with high affinity receptors for human IgE (Hu FcεRI). To measure a functional response, the invention measures a cellular response either to define the specificity of the IgE obtained from the allergic individual or to detect allergens in the environment or in a sample of interest. This invention can utilize any cultured cell that can be carried in the laboratory and that has unoccupied high affinity receptors for human IgE (i.e., FcεRI) on the surface of the cell. For example, we have used RBL SX-38 cells, which are cells which express human FcεRI at a relatively high levels, but any cell expressing sufficient Hu FcεRI to trigger a cellular response after binding of IgE and allergen can be used. This includes, but is not limited to cultured human mast cells and cultured cells of other sources that are transformed to express Hu FCεRI in a functional manner. Cell activation can be assessed by measuring degranulation, the release of various compounds from the cells or by measurement of intracellular changes. Compounds which are mediators of IgE-allergen binding include, but are not limited to, serotonin, histamine, tryptase, leukotrienes, prostaglandins and cytokines. The molecular signal that demonstrates cell activation in response to IgE and allergen binding to the Hu FCεRI can be a radioactive moiety, a fluorophore, a chromophore, a luminescent moiety, or a ligand capable of binding to a further labeled ligand partner, which may in turn be labeled with a radioactive, luminescent, fluorescent or chromophoric moiety or it may contain an enzyme substrate which is chromophoric or otherwise detectable and distinguished from unreacted substrate. It is understood in the art how to choose a compound for monitoring and to label it so as to allow detection. The labeled cells are then sensitized by incubating them with human serum under conditions which allow the binding of serum IgE to the Hu FcεRI receptors and activation of the cells so that a detectable signal is generated. Depending on the relative concentrations of specific and nonspecific IgE in the serum, additional nonspecific IgE may be added. Cells with IgE bound to the Hu FcεRI in this way are said to be sensitized.

The sensitized cells in this fashion can then be triggered by adding purified antigens (allergens), commercially available allergen extracts (ALK or other commercial source), or freshly prepared extracts comprising a substance of interest, or unknown samples which contain the substance of interest or samples suspected of containing the substance of interest. The labeled and sensitized cells are optionally further sensitized by exposure to N-ethylcarboxamido adenosine (NECA) to lower the threshold for triggering by allergen. Other substances, including deuterium oxide (D₂O) or cytochalasin B, can also be used with similar effect.

Triggering is initiated by adding allergen or goat anti-human-IgE in the present assay. Nonspecific (background) release is assessed using negative controls, including using no serum, serum with no IgE or using buffer instead of allergen extract or anti-IgE. After a suitable incubation period, the label is quantitated in the cell free supernatant. To correct for small variations in cell numbers per dish or well, the adherent cells can be detached and solubilized, for example, using a surfactant. This suspension contains the label or other detectable product associated with cells which was not previously released, and constitutes the residual value.

As specifically exemplified herein, we have labeled RBL SX-38 cells with ³H-serotonin (5HT). A fraction of the tritiated 5-HT incorporated into cellular granules is released from the cells (in the process of degranulation) following their activation. The percentage of labeled 5-HT release, where the label is a radioactive moiety, is calculated as follows: disintegrations per minute (dpm) in the supernatant divided by the sum of the dpm in the supernatant plus the dpm in the residual cells. This formula is as follows:

% release =100×(dpm in the supematant)/(dpm in the supernatant+dpm in the residual cells).

For each experiment, a positive control, activation of the cells with anti-IgE is determined. The % release with anti-IgE (usually about 30-60% of the total 5-HT) is set at 100%. Degranulation with a given allergen is then expressed as a percent of that seen with anti-IgE.

Background release (for example in response to a buffer control as trigger) is subtracted from the release triggered by the antigen/allergen of interest. Where there is at least about 10% release over background or a response that is 25% of that seen with anti-IgE, there is a positive response to that particular antigen. Where there is a positive response to this test with patient serum and a particular allergen-containing composition, the IgE-mediated response is deemed to be serious, and the patient is counseled to avoid exposure to the allergen/antigen.

Human sera are prepared from blood collected from allergic donors and non-allergic control subjects by promptly centrifuging at room temperature for a time and with sufficient g forces (for example, 10 min. at 200 g) to separate the serum from cellular elements. These sera can be frozen in aliquots and stored at −20E C. for later use. Commercially available allergenic extracts (ALK, Abello, Pharmacia or other sources), purified antigens or crude preparations of allergens can be used to trigger 5-HT release from the labeled and sensitized cells in the present system. The ability of the sensitized cells to be activated by a specific allergen is dependent upon their being sensitized with IgE from a patient sensitive to that allergen or by another naturally occurring or engineered molecule that can bind Hu FcεRI and allergen.

In the present assay methods, sera prepared from one or more individuals who are known to be extremely allergic to a particular allergen can be used as positive controls, either singly or as pooled sera. Serum(a) from individuals known not to exhibit a significant response to a particular allergen of interest can be used as negative control samples in the assay of the present invention. For negative control sera, there is typically from about 0 to about 15% release of labeled 5-HT compared to that from cells triggered by the allergen of interest. Where the patient serum has a response of at least 15% 5-HT release above background or 25% of that seen with anti-IgE in response to exposure of the labeled and sensitized cells to allergen, the response is considered positive.

By virtue of its ability to safely assess the allergic sensitivity of an individual based on the interaction of that individual's IgE with specific allergens, the present invention provides a specific allergen functional IgE (SAFE) test. Where there is a positive response in this assay, the patient is deemed to have the capacity for a potentially life threatening response to the allergen. That patient is then counseled to avoid exposure to the particular allergen or composition.

As described herein, the methods of the present invention are useful in recognizing individuals with severe responses to a particular allergen. The allergen(s) can be found in an aeroallergen (e.g., grass pollen), in a food (e.g., peanut), in an environmental material (e.g., latex), an insect venom (e.g., bee venom) or it could be a pharmaceutical compound such as an antibiotic or an analgesic. The assay can be adapted for use in a medical setting, to predict (and then prevent) dangerous reactions during treatment or it can be used to identify problematic triggers of allergic reactions of patients. The methods of the present invention can also be applied to the diagnosis of less severe IgE-mediated sensitivities as well.

The present methods are improved over the so-called RAST (radioallergosorbent) test in that the present assay is a functional assay and measures a physiological response after IgE-allergen binding. The RAST test measures only binding of IgE to allergen and does not have a functional component.

The methods of the present invention are improved over previous use of RBL SX-38 cells in that the present methods allow the assessment of individual sensitivity(ies) to single and multiple allergens, they allow detection of picogram quantities of functional allergens in the environment or in a sample, and the present methods can further be used to standardize allergenic extracts or sera containing IgE of particular specificities of interest. These cells have previously been used to simply detect allergen-induced degranulation.

The present invention further provides a functional allergen detection (FAD) test, a sensitive method for the detection of small amounts of an antigen of interest. The assay is modified so that IgE, a serum known to provide a strong response in the cell activation assay (e.g., 5-HT release assay) or another molecule (naturally occurring, engineered or synthesized) that binds to Hu FcεRI and allergen is used in conjunction with a test sample or extract of a material for which analysis for a potentially dangerous allergen is desired. If there is not significantly greater cell activation (for example, as measured by 5-HT release) in the assay containing the test sample or extract as compared with a negative control, then one concludes that the test sample or extract did not contain a detectable amount of allergen to which the IgE was known to bind. Where the cell activation (for example, as measured by release of 5-HT) was significantly greater with the test composition, one concludes that the test sample or extract contained at least one allergen to which the IgE in the assay was known to bind.

Also within the scope of the present invention are methods for standardizing an allergen extract, where the extract contains an antigen of interest. As described herein, a panel of selected sera can be used either individually or as a pool to compare one extract with a previously defined standard. Background 5-HT release and release in response to varying amounts of the extract (or dilutions thereof) are determined, so that allergen/antigen content in the extract, as measured by the ability to trigger 5-HT release in the labeled and sensitized cells, can be adjusted for consistency in the percentage of 5-HT release in the present assay in various preparations.

### BRIEF DESCRIPTION OF THE DRAWINGS

FIG. 1 is a schematic describing the basis of the present invention. The cell sketched on the top left is any cultured cell which expressed unoccupied Hu FcεRI. Serum (a) containing allergen-specific IgE is added as illustrated at top right. The IgE in the serum binds to Hu FcεRI with high affinity, and excess (unbound) IgE is washed away (bottom left). The Allergen is then added, crosslinking the IgE—Hu FcεRI complexes and leading to cell activation which can be measured in a variety of ways (bottom right).

FIG. 2 shows that the binding of human IgE to RBL SX-38 cells which express the human FcεRI on the surface exhibits saturation kinetics. RBL SX-38 cells are the prototypic cells expressing Hu FcεRI. Serum from an allergic donor was diluted in buffer containing 0.1% bovine serum albumin, 25 mM disodium PIPES, 100 mM NaCl, 5 mM KCl, 0.4 mM MgCl₂, 1 mM CaCl₂, and 5.6 mM glucose (pH 7.0) to contain IgE at the concentration shown. After an overnight incubation, cells were harvested, washed and stained with biotinylated anti-IgE and then FITC-labeled avidin. Stain intensity was determined by fluorescent activated cell analysis and expressed as a fold-increase of the base-line fluorescence intensity.

FIG. 3 shows, by using serum from selected allergic individuals, that the methods of the present invention is very sensitive, i.e., cells can be triggered with markedly dilute extracts of antigens. The experiment shown utilized RBL SX-38 cells sensitized with serum from allergic individuals. Cells were sensitized with serum from donors who were allergic to grass (left) or peanut (right). After excess IgE was washed away, the cells were triggered with anti-IgE (3 μg/ml) or diluted extracts of grass pollen (for serum from a grass-allergic patient, left) or extracts of peanut (for the serum from a peanut-allergic patient, right). Commercially available extracts that are used without dilution for skin tests in the medical practice of Allergy and Immunology were added at the dilutions shown; 1 M refers to a dilution of 1:1 million and 100 M is a dilution of 1:100 million.

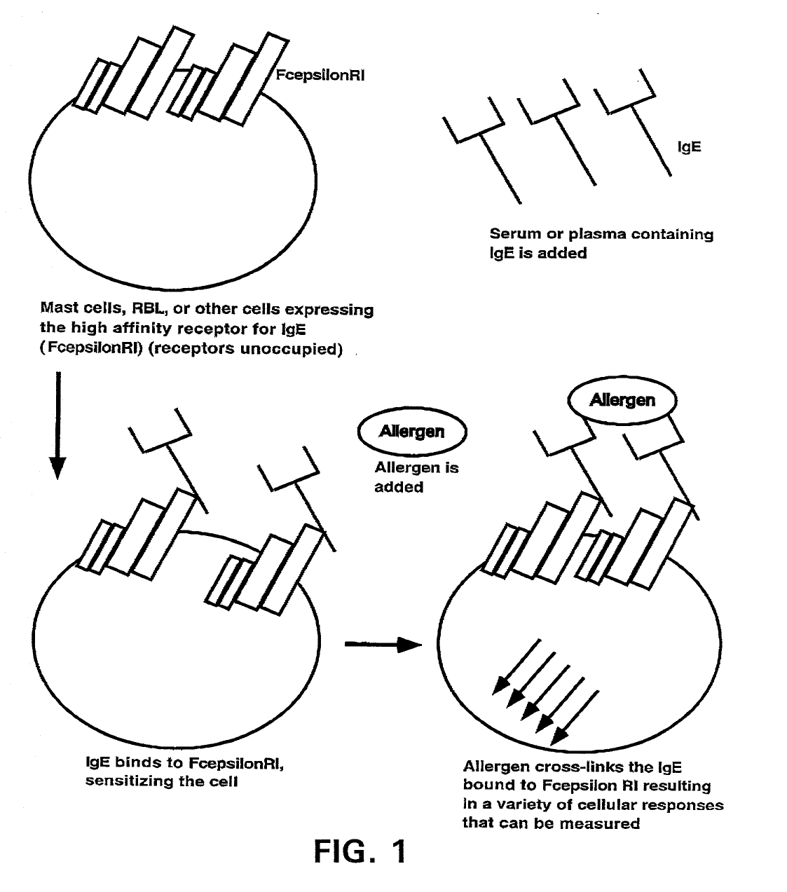

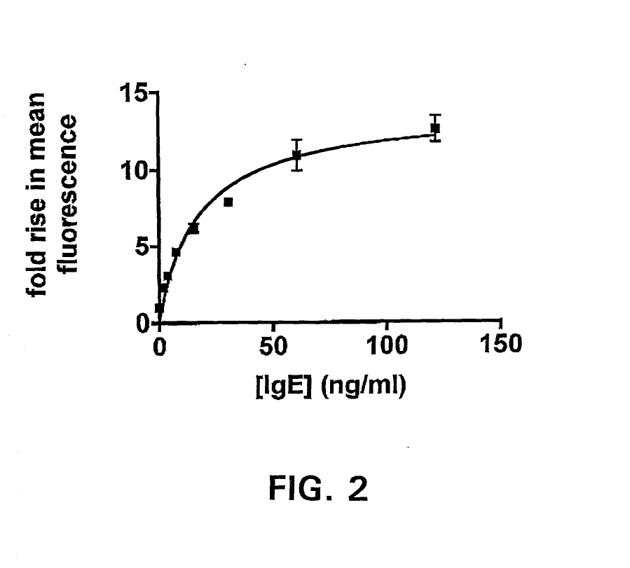

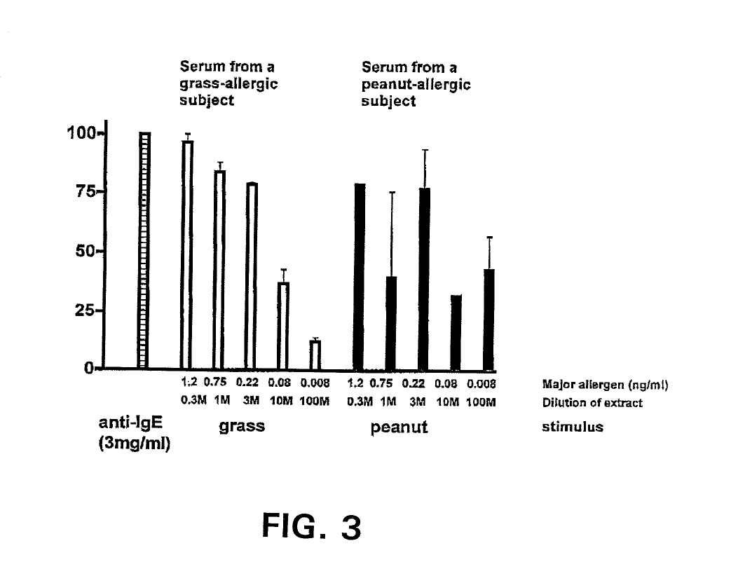

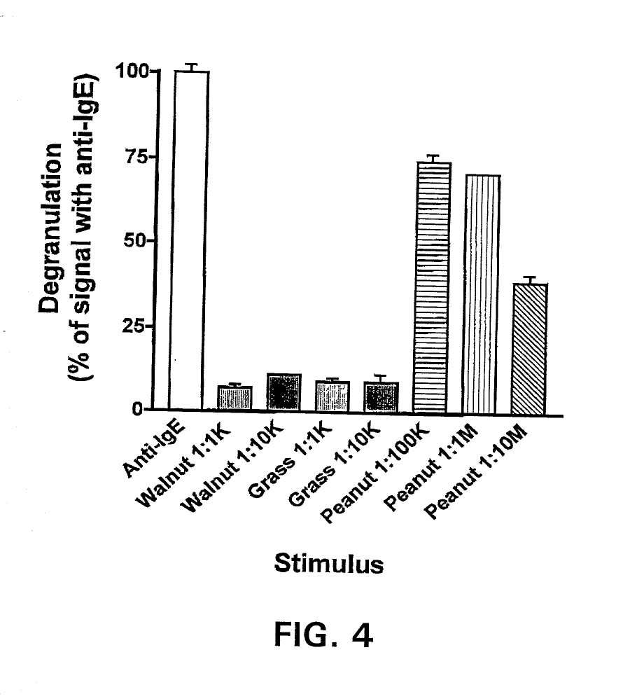

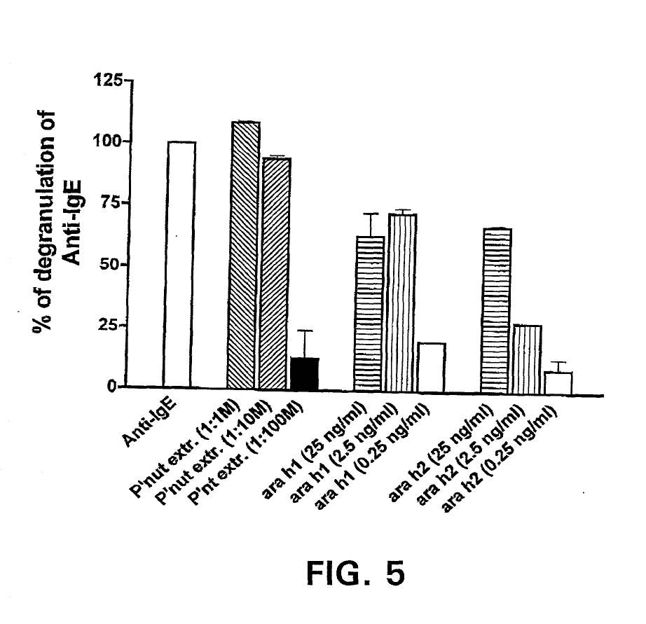

The release with anti-IgE (positive control) is shown as 100% on the far left (hatched bars) and release with the extracts is shown as a percentage of the release with anti-IgE. It can be seen that a 1:3 million dilution of either grass pollen extract (for the grass-sensitive patient) and a 1:3 million dilution of the peanut extract (for the peanut sensitive patient) gives cell activation that is similar to that seen with the positive control, anti-IgE. The grass pollen extract used in the present experiments contains about 280 pg/ml of the major grass allergen in a 1:3×10⁶ dilution. The peanut extract contains about 280 pg of major allergen (ara h1 plus ara h2) in a 3×10⁶ dilution.

FIG. 4 demonstrates that the invention is highly specific. RBL SX-38 cells were sensitized with serum from a peanut allergic donor who had no detectable IgE to soy, walnut or timothy grass pollen. After excess IgE was removed by washing, the cells were contacted with anti-IgE (3 μg/ml) or diluted extracts of peanut, soy, walnut or timothy grass pollen as indicated. Extracts were added at the dilutions shown; 1 K refers to 1:1000, and 1 M refers to a dilution of 1:1 million. The maximum net degranulation, as measured by detectable 5-HT release, in response to anti-IgE (positive control) is shown as 100%. 1:10⁶, and 1:1×10⁶ dilutions of peanut extract, and three dilutions (1:1000, 1: 10⁶, and 1:1×10⁶ dilutions) of soy, walnut and timothy grass pollen allergens are shown as percentages of the maximal release with anti-IgE. These results demonstrate the specificity of the assay. Studies with patients known to have problematic responses to a particular allergen established that a positive response in the present method reflects an allergic response of concern in the patient tested. Some previously used assays five positive responses even where the response of the patient to the allergen is mild and of no particular medical concern. The grass pollen extract used in the experiment shown contains about 280 pg/ml of the major grass allergen in a 1:3×10⁶ dilution. The peanut extract used in the experiment shown contains about 280 pg/ml of the major allergens (ara h1 and ara h2) in a 1:3×10⁶ dilution. The equality of the amounts of major allergens in these extracts was unexpected and coincidental.

FIG. 5 further illustrates the extreme sensitivity of the methods of the present invention and demonstrates its ability to detect specific allergens. In this experiment, cells were sensitized with serum from a peanut allergic individual and triggered with peanut extract (at the dilutions shown or purified major peanut allergens, ara h1 or ara h2 (at the concentrations shown) (described in U.S. Pat. Nos. 5,973,121 and 5,558,869; gift from Wesley Burks and Gary Bannon, University of Arkansas).

### DETAILED DESCRIPTION OF THE INVENTION

As used herein, mediators of IgE-allergen binding are molecules which either are released from FcεRl-expressing cells as a result of degranulation (triggering) or otherwise released or expressed via de novo protein synthesis as a result of the signal generated within the cell due to the cross linking by allergen bound to IgE on the cell surface, which IgE is bound to the Fc RI receptors on the cell surface. Such mediators of IgE-allergen binding include, but are not limited to, histamine, serotonin (5-hydroxytryptamine, 5-HT), certain leukotrienes, certain prostaglandins, certain cytokines (e.g., interleukins 4 and 6 and tumor necrosis factor alpha).

Reporter gene constructs can be incorporated into the test cells so that gene expression as a result of triggering and binding of allergen to IgE bound to the cognate receptors on the cell surface. Reporters can include enzymes whose activities can be measured (beta-galactosidase, betalactamase, beta-glucuronidase and the like), fluorescent green protein and a number of others well known to the art. The coding sequence for the reporter is operably linked to a NF-KB or AP-1 transcriptional regulatory sequence.

Cell types useful in the practice of the present invention include those which express the high affinity IgE receptors (Hu FcεRI) on the cell surface and for which there is a mechanism for evaluation of allergen binding. Human basophils and mast cells contain histamine and other mediators and can be triggered to release these mediators (e.g., 5-HT) and generate other detectable signals. Human basophils with unoccupied receptors can be obtained from selected non-allergic donors and can be sensitized by methods as described herein or modified by one of skill in the relevant art. However, these cells are only available from selected donors, are short-lived and cannot be depended upon as a consistent and reproducible bioassay over time. Thus, human basophils are inferior to the cells envisioned for use in this invention and for which the RBL SX-38 cells are a prototype. Human mast cells with unoccupied receptors can be cultured from human bone marrow, human stem cells and from patients with mast cell diseases such as mastocytosis. These cells can be carried in tissue culture for substantial periods of time and are compatible for use as in the practice of the present invention. Rodent mast cells and basophils express rodent FcεRI and contain both histamine and 5-HT. However, these and other cell types can be engineered to express the human FcεRI and further engineered to contain a reporter gene system in which expression of the reporter gene is triggered by allergen binding to the cell bound IgE molecules. Examples of suitable promoters for the reporter gene constructs include the NfκB and AP-1 promoters. Reporter proteins can include, without limitation, luciferase, aequorin, chloramphenicol acetyltransferase, β-glucosidase, β-galactosidase, β-glucuronidase and β-lactamase, among others. Techniques for monitoring and quantitating expression of these proteins are well known to the art, and the choice of vectors and methods for introducing heterologous DNA into a eukaryotic cell, especially a mammalian cells, including human cells, are well known to the art. Other vector systems for the expression of this receptors can be used to give greater receptor number on the cell surface, for example using a retrovirus vector such as LNCX which can be constructed to express the human FcεRI receptor coding sequence. Recombinant human FcεRI receptor beta subunit and coding sequences are described in U.S. Pat. Nos. 5,807,988 and 5,770,396. U.S. Pat. No. 4,962,035 describes recombinant expression of the alpha subunit of rat mast cell FcεRI receptor. GenBank contains several IgE receptor coding sequences as well; see, e.g., Accession Nos. X 06948, NM 002001, D10583, Z29585, M15059, J03605, P12319 and gi 1 19865. Rat basophil leukemia RBL 2H3 cells are available from American Type Culture Collection, Manassas, Va.

As specifically exemplified in the present disclosure, RBL SX-38 cells are used as the cells which express the human FcεRI receptors on the cell surface and which trigger response to binding of antigen (allergen) to IgE molecules which in turn have bound to the FcεRI receptors. RBL SX-38 cells are rat basophile leukemia cells of the RBL-2H3 line that have been transfected in a stable fashion with DNA encoding human FCεRI. These cells were obtained from Jean Pierre Kinet, and equivalent cells expressing the human IgE receptors can be produced using published procedures and sequences and readily available cultured cells. These cells have been used to demonstrate the methods of the present invention, but RBL 48 cells (prepared by Jean Pierre Kinet in collaboration with J. Kochan of Hoffinan LaRoche) can also be used. The RBL SX-38 cells have provided better results than the RBL 48 cells to date.

The present invention provides a specific allergen functional IgE (SAFE) test. This is a reliable and safe test for important IgE-mediated immune responses. The present in vitro assay can replace skin testing which relies on percutaneous or intradermal administration of allergen-containing materials. Intradermal skin testing can be dangerous to the patient in that there is the risk of a life-threatening allergic response in a patient who is extremely sensitive to an allergen administered. In addition, there is no need for the patient to discontinue the use of allergy medications before or during the testing, as required for percutaneous and intradermal skin testing. The physician does not need to stock a bank of allergen extracts because blood as serum can be sent to a medical laboratory for testing. The physician or his/her medical assistants need only be trained in phlebotomy and not in skin test application or interpretation. Further, the patient does not have to endure the discomfort associated with traditional skin testing procedures.

Another test used for the detection of sensitivity (IgE-mediated) to a particular allergen is test administration directly to a mucosal surface of the patient being tested (e.g., nasal or bronchial allergen challenge). Again, this methodology places the patient at risk for a dangerous systemic immune response and for discomfort associated with a positive response. Furthermore, it is likely that the patient would need to discontinue allergy medication for some prior to the mucosal testing.

Another available test for allergen responsiveness is the RAST (radioabsorbant) test, recently further developed as the ImmunoCap assay. In these assays, allergens are linked to a solid support. The present methods of the present invention are improved over the RAST assay in that the present methods are functional tests and use allergens in their native state. Where allergens are coupled to a solid support, the allergens might be at least partially denatured and native conformational epitopes of the allergen may be lost.

The present invention further provides a functional allergen detection (FAD) test. Because the amount of 5-HT released in the specifically exemplified assay methods of the present invention is dependent on the amount of specific antigen bound when there is saturating specific IgE bound to the labeled and sensitized cells, the present assay can be used to detect and/or quantify the amount of a particular antigen or allergen present in a test sample. Background release of 5-HT is determined in an assay in which buffer is substituted for the positive allergen-containing control or the test sample. The background release is subtracted by the release in response to the test samples. The test sample(s) is that in which the presence, absence or amount of allergen (or antigen) is being determined. The specific IgE is typically obtained from individual or pooled sera from patients known to be sensitive to a particular antigen or allergen extract, which may or may not contain only one antigenic component. For example, peanut extract contains many individual compounds and thus, many potential immunologically distinct antigens.

Food products can be tested for the presence of allergens, and appropriate choice of the IgE source allows the detection of a single allergenic compound (such as penicillin or the Ara hi peanut allergen) or the presence of a particular material (such as peanut-derived materials without limitation to a particular component) in a product. The present method could be used to detect the presence of a particular allergen in heterologous material, for example, the presence of a peanut allergenic component in a corn or soybean product; such detection could be evidence of genetic engineering of crops or deliberate or accidental contamination of a food product with an allergenic substance. Similarly, the presence of penicillin in either a food or medical product could be detected using the appropriate serum (IgE) source. This assay can be used to screen specifically bred or genetically altered foods for persistence of allergenicity without the need for in vivo testing.

In addition, because the percent 5-HT release over background release triggered in response to a particular antigen cognate to IgE bound to labeled and sensitized cells is directly related to the quantity of the antigen, the present methods can be adapted for the standardization of an allergen extract. Varying amounts of extract protein are added to the labeled and sensitized cells, and the percent release is determined. The dilution of the extract or the concentration of allergen in the extract can be adjusted so that the quantity to be added in an assay of the present invention is in the range over which the response of 5-HT release is proportional to the dose of allergen added to the assay. The functional concentration of allergen in the extract is more important and more relevant than simply adjusting the protein or carbohydrate concentration in the extract to some particular value or by assaying the immunoreactivity of materials in the extract to IgE in a RAST, ELISA or RAST inhibition assays.

As specifically exemplified in the present disclosure RBL SX-38 cells are used as the cells which express the human FcεRI receptors on the cell surface. RBL cells are rat basophilic leukemia cells, and they have been transfected to express noted human receptors. Naturally occurring human mast cells that can be cultured or other cultured cells expressing the human FcεRI can be used. Vector systems for the stable expression of a protein of interest are well known to the art. Vectors encoding the human IgE receptor can be used to give greater receptor number on the cell surface, for example using a retrovirus vector such as LNCX which can be constructed to express the human FcεRl receptor coding sequence, desirably under the regulatory control of a strong promoter. Recombinant human FcεRI receptor beta subunit and coding sequences are described in U.S. Pat. Nos. 5,807,988 and 5,770,396. U.S. Pat. No. 4,962,035 describes recombinant expression of the alpha subunit of rat mast cell FcεRI receptor. Numerous human IgE receptor sequences are also available on GenBank.

Fourteen separate serum samples from peanut-sensitive patients and three sera from grass-sensitive patients have been tested in the method of the present invention.

FIG. 4 shows that the SAFE test is highly specific. The maximum net degranulation, as measured by 5-HT release, in response to anti-IgE (positive control) is shown as 100%, 1:10⁶ and 1:10×10⁶ dilutions of peanut extract, and three dilutions (1:10³, 1:10×10³ and 1:100×10³) of soy, walnut and timothy grass pollen allergens are shown as a percentage of the maximal release with anti-IgE. These results demonstrate the specificity of the assay. Studies with patients known to have problematic responses to a particular allergen established that a positive response in the present method reflects an allergic response of concern in the patient tested. Some previously used assays give positive responses even where the response of the patient to the allergen is mild and of no particular medical concern.

As disclosed herein, the present assay provides methods which have the advantage of a functional assay in that it is not total IgE in serum being measured or even IgE which binds a particular antigen. Rather, the strength of the 5-HT releasing response and thus the ability of the IgE from the patient to be functionally cross-linked by allergen is measured in response to an allergen/antigen of interest. The test avoids danger and discomfort to the patient in that it is done in ex vivo, and there are economic advantages in that physicians or their assistants need only draw a blood sample to be sent to a testing laboratory which can service a relatively large number of physicians. The individual physicians and their assistants do not need to stock antigens and extracts of antigens nor do they need to be trained in assay performance and interpretation.

As used herein, labeled 5-HT, histamine or other mediator of IgE-allergen binding, carries a detectable moiety which allows detection and quantification. The detectable moiety can be a radioactive atom (e.g., ³H or ¹⁴C), a fluorescent, chemiluminescent or chromophoric moiety or other detectable label or material potentially produced by RBL cells as they now exist or as they can be genetically altered as currently known to the art. The requirement in the present invention is that the cells expressing human IgE receptor are a consistent bioassay, they can be sensitized with IgE from the serum(a) of allergic individuals and can be triggered with specific allergen.

The binding of the antigen/allergen cross links adjacent IgE bound to receptors and effects the activation of the cells.

Monoclonal or polyclonal antibodies specifically reacting with a protein or other molecule of interest can be made by any of a number of methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Springer-Verlag, New York, N.Y.; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

All references cited in the present application are incorporated by reference herein to the extent that there is no inconsistency with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1

### Maintenance of RBL SX-38 Cells in Tissue Culture

RBL SX-38 cells which express the α, β and γ subunits of the human FcεRI receptor (Weigand et al. (1996) Journal of Immunology 157, 221-230; Takebe et al. (1988) Mol. Cell. Biol. 8, 466; Lin et al. Science 249, 677; U.S. Pat. Nos. 5,807,988; 5,770,396; 4,962,035) are grown in Eagle?s Minimal Essential Medium (Gibco, Grand Island, N.Y.) with 10% fetal calf serum (HyClone Laboratories, Inc., Logan, Utah), 1% penicillin/streptomycin (Gibco), (culture medium) and optionally, 1.2 mg/ml G418 (geneticin, Invitrogen, Carlsbad, Calif.). Every other passage, the G418 is increased to 2.4 mg/ml to maintain a high level of expression of human FcεRI. At each passage, cells are detached using 0.05% Trypsin-EDTA 0.53 mM (Gibco) for 6 minutes at 37 EC, washed once with culture medium (CM), and centrifuged at 1000 rpm for 5 minutes. Following resuspension in fresh medium, cells are aliquoted for passage or for experiments.

### Example 2

### Preparation of RBL SX-38 Cells for Experiments

Two days prior to each experiment, cells from a culture flask in which the cells are grown to confluence are detached, washed and replated at 5×10⁵ cells/ml. Either 35 mm dishes or 48-well plates are used, depending on the experiment. The CM used to resuspend the pellet contains 1 FCi/ml tritiated 5-hydroxytryptamine (serotonin) (³H-5-HT, NEN Life Science Products, Inc., Boston, Mass.) to label the cells. The [³H]5-HT is taken up and stored in granules; cells with [³H]5-HT in the granules are said to be labeled. After resuspending the cells in the CM containing [³H]5-HT and aliquoting them into dishes or wells, human serum (from an allergic donor to be tested) is added to sensitize the cells. See also Weigand et al. (1996) supra for discussion of RBL SX38 cells.

Typically, 1.25×10⁵ cells in 225 μl of[³H]5-HT-containing CM are added to each well of a 48-well plate. Then, 25 μl of serum is added to each well to achieve a final concentration of 10% serum and 5×10⁵ cells/ml. The cells are incubated for 40-48 hours at 37 C. with 5% CO₂ to allow the binding of IgE to Hu FcεRI receptors to reach equilibrium and to allow the cells to enter a quiescent state. Depending on the relative concentrations of specific and nonspecific IgE, additional nonspecific IgE may be added. Cells with IgE bound to Hu FcεRI in this way are said to be sensitized. RBL SX-38 cells ?labeled? and ?sensitized? in this fashion can then be triggered by adding purified antigens, commercially available allergen extracts (ALK), or freshly prepared extracts.

### Example 3

### RBL SX-38 Cell Triggering by Specific Antigens

The labeled and sensitized RBL SX-38 cells are washed twice with PIPES buffer containing 0.1% bovine serum albumin, 25 mM disodium PIPES, 100 mM NaCl, 5 mM KCl, 0.4 mM MgCl₂, 1 mM CaCl₂, and 5.6 mM glucose (pH 7.0). After washing, 100 μl of this PIPES buffer, containing 0.1 mM NECA (Sigma Chemical Co., St. Louis, Mo.) in DMSO added (final DMSO dilution 1:1000), is placed in each well and the plates are placed in a 37 C. shaking water bath for 5 minutes. The NECA lowers the threshold for triggering. Other substances, such as 30% D₂O or 5 μg/ml cytochalasin B, can also be used with similar effect.

Triggering is initiated by adding 5 μl of allergenic extracts (diluted to the appropriate concentration) or goat anti-human-IgE (3 μg/ml). Cells are returned to the 37 C. shaking water bath. Non-specific background release is assessed using negative controls, which have 5 μl PIPES buffer added instead of extract or anti-IgE. After 45 minutes, the plates are then removed and the tritium content of a 50 μl aliquot from the supernatant of each sample, in 5 ml of scintillation fluid, is determined. In selected experiments, the supernatants are centrifuged to demonstrate that the release of[³H]5-HT is not due to detachment of cells. To correct for small variations in cell numbers per dish or well, the adherent cells are solubilized using 250 μl of 1% Triton X- 100 detergent (Pierce Chemical Co., Rockford, Ill.) at room temperature for 30 minutes, followed by scraping. This suspension contains the [³H]5-HT associated with adherent cells which was not previously released, and constitutes the residual.

### Example 4

### Calculation of Percentage Release

The percentage of[³H]5-HT release is calculated as follows: disintegrations per minute (dpm) in the supernatant divided by the sum of the dpm in the supernatant plus the dpm in the residual cells. This formula is as follows: % release=100×(dpm in the supernatant)/((dpm in the supernatant+dpm in the residual cells)).

### Example 5

### Preparation of Human Sera

For preliminary experiments, human sera were prepared from blood collected with informed consent from allergic donors and non-allergic control subjects under a protocol approved by the Colorado Multiple Institutional Review Board. Approximately 20 ml of blood were drawn into plain (“red-top”) tubes (Becton-Dickinson, Franklin Lakes, N.J.) and promptly centrifuged at 2000 rpm at room temperature for 10 minutes to separate the serum from cellular elements. These sera were then freshly frozen in aliquots and stored at −20 C. for later use. With our current procedures, one standard red top tube (yielding approximately 3 ml of serum) is sufficient to test for 6 allergens. Sera from a number of known sensitive patients are desirably pooled for use in certain of the assays of the present invention. Plasma may be substituted for serum.

It has recently been observed that sera from approximately one in three human donors contain naturally occurring activities (probably IgG antibodies) that kill and/or inactivate the RBL SX38 cells and thus interfere with the utilization of these sera in the practice of the invention. The interfering activity is removed by pre-incubating the sera with RBL-2H3 cells (which do not express the Hu FCεRI).

To collect IgE from patients who are highly sensitive to an allergen of interest, especially for use in assays to detect allergens which trigger a severe IgE-mediated response, plasmapheresis can be used. Informed consent in accordance with standard practice before the Colorado Multiple Institutional Review Board is obtained before biological material is collected. After collection, plasma can be defibrinated and pooled to create a relative standard for replicate assays. Pooled plasmapheresis from 5 selected donors is sufficient for about 15,000 to about 45,000 detection assays using the methods described herein for the 48 well plates. Adaptation of the methods to use 96 well plates can result in doubling the number of assays possible with a given volume of treated plasma.

Where the present assay is adapted for detection of a particular antigen, the cells are labeled and sensitized (using sera from patient(s) known to be responsive to the antigen or allergen of interest) as described above. Then, as a positive control, a sample known to contain the antigen is added to trigger 5-HT release from the cells. Other aliquots of the labeled and sensitized cells are combined with the varying amounts sample being tested for the presence of the antigen of interest. Appropriate amounts of known allergen can be used to generate a standard curve for % 5-HT release. Buffer added to the labeled and sensitized cells serves as a negative control (background release). Then, release of the labeled 5-HT is determined in the positive control assay and in the test sample assays. Where there is at least about 10% release of the labeled 5-HT in the test sample over that of the background, the test is deemed positive, and the amount of the allergen/antigen present in the test sample can be estimated by reference to the standard curve generated with the known antigen samples.

### Example 6

### Preparation of Extract Dilutions

Commercially available allergenic extracts (ALK, -Abello, Pharmacia or other commercial source) or other extracts to be standardized are diluted using serial 100-fold dilutions into PIPES buffer to reach appropriate triggering doses (e.g., 1:100,000 of BAU timothy grass extract or 1:1,000,000 of 1:10 w/v peanut extract). Alternatively, purified antigens can be used in this assay.

What is claimed is:

1. A method for detecting an allergen of interest targeted by IgE in a test composition, said method comprising the steps of:
(a) contacting cells which express an IgE receptor protein on the surface of the cells with a detectable mediator of IgE-allergen binding and with a composition comprising IgE which binds an allergen of interest to produce sensitized cells;
(b) contacting the sensitized cells of step (b) with a test composition;
(c) detecting detectable mediator released by the cells contacted with the test composition,
whereby allergen is detected when there is greater release of the detectable mediator by the cells contacted with the test composition than by a comparison not contacted with the test composition.

2. The method of claim 1 wherein the IgE receptor protein is human FcεRI.

3. The method of claim 2 wherein the cells which express FcεRI are RBL SX-38 cells.

4. The method of claim 1 wherein the detectable mediator is detectable 5-hydroxytryptamine.

5. The method of claim 4 wherein the detectable 5-hydroxytryptamine is [³H]-5-hydroxytryptamine.

6. The method of claim 1 wherein the composition comprising IgE is serum.

7. The method of claim 1 wherein the detectable mediator is histamine, a leukotriene, a prostaglandin, a cytokine, tryptase, interleukin-4, interleukin-5, or tumor necrosis factor alpha.

8. A method for diagnosing IgE reactivity to an allergen of interest in a patient, said method comprising the steps of:
(a) preparing cells which express an IgE receptor protein on the surface of the cells to produce a detectable mediator of IgE-allergen binding to produce sensitized cells;
(b) contacting the sensitized cells of step (b) with a composition comprising an allergen of interest; and
(c) detecting detectable mediator released by the cells contacted with the test composition,
whereby IgE reactivity to the allergen of interest is detected in the patient when there is greater release of detectable mediator by the cells contacted with the composition comprising the allergen of interest than by a comparison not contacted with the serum of the patient.

9. The method of claim 8 wherein the IgE receptor protein is human FcεRI.

10. The method of claim 9 wherein the cells which express human FcεRI are RBL SX-38 cells.

11. The method of claim 8 wherein the detectable mediator is detectable 5-hydroxytryptamine.

12. The method of claim 11 wherein the detectable 5-hydroxytryptamine is [³H]-5-hydroxytryptamine.

13. The method of claim 8 wherein the composition comprising IgE is serum.

14. The method of claim 8 wherein the detectable mediator is histamine, a leukotriene, a prostaglandin, a cytokine, tumor necrosis factor alpha or tryptase.

15. A method for standardizing a composition comprising an allergen of interest, said method comprising the steps of:
(a) contacting cells, said cells expressing an IgE receptor protein on the surface of the cells, with a composition comprising a standardized IgE known to bind an allergen of interest and with a detectable mediator of IgE-allergen binding to produce sensitized cells;
(b) separately contacting the sensitized cells of step (b) with a test composition comprising comprising the allergen of interest in unknown amount and in an amount known to be sufficient to provide a predetermined release of the detectable mediator; and
(c) detecting the detectable mediator released by the cells contacted with the test composition and with the composition comprising the allergen of interest sufficient to provide a predetermined release of the detectable mediator;
whereby the test composition is adjusted to provide the allergen of interest in an amount sufficient to provide a predetermined release of the detectable mediator.

16. The method of claim 15 wherein the IgE receptor protein is human FcεRI.

17. The method of claim 16 wherein the cells which express human FcεRI are RBL SX-38 cells.

18. The method of claim 15 wherein the detectable mediator is detectable 5-hydroxytryptamine.

19. The method of claim 18 wherein the detectable 5-hydroxytryptamine is [³H]-5-HT.

20. The method of claim 15 wherein the composition comprising IgE is serum.

21. The method of claim 15 wherein the detectable mediator is histamine, a leukotriene, a prostaglandin, a cytokine, interleukin-4, interleukin-5, tryptase or tumor necrosis factor alpha.

22. A method for standardizing a composition comprising IgE which binds an allergen of interest, said method comprising the steps of:
(a) separately contacting cells, said cells expressing an IgE receptor protein on the surface of the cells, with a composition comprising IgE known to bind an allergen of interest in an amount sufficient to produce a predetermined release of 5-hydroxytryptamine and with a test composition comprising IgE known to bind the allergen of interest and with detectable 5-hydroxytryptamine (5-HT) or allowing the cells to produce a detectable mediator of IgE-allergen binding to produce sensitized cells;
(b) separately contacting the sensitized cells of step (b) with a test composition comprising the allergen of interest in an amount sufficient to provide a predetermined release of 5-HT or other detectable mediator of IgE-allergen binding after binding with the composition comprising IgE known to bind the allergen of interest;
(c) detecting detectable 5-HT or other mediator of IgE-allergen binding released by the cells contacted with the test composition and with the composition comprising the allergen of interest sufficient to provide a predetermined release of 5-HT;
whereby the test composition comprising IgE which binds the allergen of interest is adjusted to provide the IgE in an amount sufficient to provide a predetermined release of detectable 5-HT or other detectable mediator of IgE-allergen binding.

23. The method of claim 22 wherein the IgE receptor protein is human FcεRI.

24. The method of claim 22 wherein the cells which express human FcεRI are RBL SX-38 cells.

25. The method of claim 22 wherein the detectable mediator is detectable 5-hydroxytryptamine.

26. The method of claim 25 wherein the detectable 5-hydroxytryptamine is [³H]-5-hydroxytryptamine.

27. The method of claim 22 wherein the detectable mediator is histamine, a leukotriene, a prostaglandin, a cytokine, interleukin-4, interleukin-5, tryptase, or tumor necrosis factor alpha.

28. The method of claim 22 wherein the composition comprising IgE is serum.

29. A method for monitoring IgE-allergen binding on the surface of a cell expressing human FcεRI, wherein the cell has been transfected to express a reporter coding sequence operably linked to an AP-1 or an NfκB transcription regulatory sequence, said method comprising the steps of:
(a) contacting the cell with a composition comprising IgE under conditions allowing binding of the IgE to human FcεRI on the surface of the cells; (b) contacting the cells of step (a) with an allergen which binds specifically to the IgE under conditions allowing binding of allergen to IgE; (c) culturing the cells of step (b) for a time and under conditions allowing expression of the reporter coding sequence in the cells to which allergen has bound to IgE, which in turmn, is bound human FcεRI on the surface of the cells; and (d) detecting expression of the reporter,
whereby binding of allergen to IgE is detected by expression of the reporter at a level greater than observed in the absence of IgE or in the absence of allergen.

30. The method of claim 29 wherein the reporter coding sequence encodes a chloramphenical acetyl transferase, a β-galactosidase, a β-glucuronidase, a β-lactamase or a fluorescent protein.
